(19) United States
(12) Patent Application Publication
(10) Pub. No.: US 2012/0029226 A1
(43) Pub. Date: Feb. 2, 2012

(54) METHOD FOR THE SYNTHESIS OF CHIRAL ALPHA-ARYL PROPIONIC ACID DERIVATIVES

(76) Inventors: Bernardus Kaptein, Sittard (NL); Elias Vlieg, Maiden (NL); Willem Lieuwe Noorduin, Nijmegen (NL)

(21) Appl. No.: 13/148,123

(22) PCT Filed: Feb. 4, 2010
(86) PCT No.: PCT/EP2010/051347

§ 371 Date: Oct. 17, 2011

(30) Foreign Application Priority Data
Feb. 6, 2009 (EP) 09152244.1

(51) Int. Cl. C07C 67/00; C07C 57/58; C07C 59/64; C07C 51/347; C07C 51/09

(52) U.S. Cl. 560/56; 562/492; 562/466

(57) ABSTRACT

The present invention provides a method for the synthesis of optically pure α-aryl propionic acid derivatives comprising subjecting the corresponding racemic α-aryl propionic acid derivatives to high sheer or impact forces, such as grinding.

### FIELD OF THE INVENTION

The present invention relates to a process for deracemizing α-aryl propionic acid derivatives by means of high sheer or impact forces.

### BACKGROUND OF THE INVENTION

The synthesis of enantiomerically pure molecules is of substantial practical importance, especially for pharmaceutical compounds that are increasingly registered in enantiomerically pure forms. Crystallization is an attractive option to obtain enantiomerically pure materials, as Louis Pasteur demonstrated by manually separating enantiomorphous crystals of a tartrate salt (L. Pasteur, C. R. Hebd. Séanc. Acad. Sci. Paris 1848, 26, 535). Resolution by crystallization can be further improved by racemizing the unwanted enantiomer. Combining crystallization and solution racemization results in a so-called total ‘spontaneous resolution’ (E. Havinga, Biochem. Biophys. Acta 1954, 13, 171). For this, enantiopure seeds are introduced in a clear supersaturated solution in which racemization takes place. These seeds grow further, resulting in an increasing amount of enantiopure solid material, until the solution is depleted. To reduce the nucleation rate of the undesired enantiomer, optionally the supersaturation can be lowered by introducing many secondary nuclei of the desired enantiomer through stirring (D. K. Kondepudi, R. J. Kaufman, N. Singh, Science 1990, 250, 975-977; J. M. McBride, R. L. Carter, Angew. Chem. Int. Ed. 1991, 30, 293). In principle, all chiral material that is crystallized can be converted into the desired enantiomer implying a theoretical yield of 100% in the solid phase. However, a drawback is that crystallization conditions, such as temperature, need to be controlled carefully in order to prevent the unwanted enantiomer from nucleating. This leaves room for more robust methods for the resolution of racemates such as the α-aryl propionic acid derivatives of the present invention which are valuable constituents of non-steroidal anti-inflammatory drugs.

### DETAILED DESCRIPTION OF THE INVENTION

In the context of the present invention, the term “grinding” refers to the mechanical treatment of solids such as crushing, pulverizing, or reducing to smaller particles by friction, for instance by rubbing between two hard or abrasive surfaces. Grinding can be effected by milling, shaking, stirring or ultrasound, optionally in the presence of particles such as beads of inert materials such as glass, ceramic, quarts, diamond, sand, metals and the like.

The term “hydrolysis” refers to a process used to convert an ester or an amide to its substituent carboxylic acid and alcohol or amine, respectively. In the context of the present invention, said hydrolysis can be any process known to the skilled person such as reaction with base or acid, or by chemicals that are particular suitable to remove a specific carboxylic acid protecting group.

The term “insoluble” refers to particles that are substantially insoluble in the reaction mixture. Substantially insoluble means solubility below 0.01 g·kg⁻¹, preferably below 0.0001 g·kg⁻¹, most preferably below 0.000001 g·kg⁻¹.

Deracemization using crystallization under abrasive grinding and near-equilibrium conditions was disclosed for three amino acid derivatives, namely N-(2-methylbenzylidene)phenylglycine amide (W. L. Noorduin, T. Izumi, A. Millemaggi, M. Leeman, H. Meekes, W. J. P. van Enckevort, R. M. Kellogg, B. Kaptein, E. Vlieg, D. G. Blackmond, J. Am. Chem. Soc. 2008, 130, 1158), N-(4-chlorobenzylidene)phenylalanine methyl ester (B. Kaptein, W. L. Noorduin, H. Meekes, W. J. P. van Enckevort, R. M. Kellogg, E. Vlieg, Angew. Chem. Int. Ed. 2008, 47, 7226) and aspartic acid (C. Viedma, J. E. Ortiz, T. de Torres, T. Izumi, D. G. Blackmond, J. Am. Chem. Soc. 2008, 130, 15274). This is a remarkably simple and much more reliable technique to reach an enantiomerically pure end state for these specific products. Unfortunately the conglomerate behavior of a compound is unpredictable and so it cannot be generalized as to whether or not the enantiomers of a given compound will form separate crystals or not. All that is generally accepted is that such conglomerates in racemates occur in only 5-10% of all cases (A. Collet, Enantiomer 1999, 4, 157, confirming similar estimates cited in that document).

In a first aspect of the present invention, it was surprisingly found that deracemization under grinding conditions is applicable to certain substrates of a structure that is chemically, electronically and morphologically quite different from the earlier disclosed amino acid derivatives, namely α-aryl propionic acid derivatives. Accordingly, the present invention provides a method for the synthesis of an α-aryl propionic acid derivative of general formula (1)

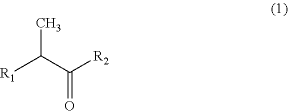

having an enantiomeric excess (e.e.) of from 50 to 99.99%, preferably of from 90 to 99.99%, wherein R₁ is substituted or unsubstituted biphenyl, naphthyl, phenyl or thienyl and wherein R₂ is an amide or OR₃ with R₃ being a carboxyl protecting group, a substituted or unsubstituted amine cation or a metal cation. The method comprises subjecting a compound of general formula (1) wherein R₁ and R₂ are as defined above having a low e.e. to mechanical processing. A low e.e. is an e.e. of from 0 to 50%, preferably of from 0.1 to 30%. The method requires at least part of said compound of general formula (1) having a low e.e. to be present in the solid state and part of said compound of general formula (1) having a low e.e. to be present in solution in a solvent. In one embodiment said latter low e.e. is equal or close to zero, i.e. racemic. The system that this mixture results in is referred to as slurry. Preferably the solvent is a solvent in which racemization of the compound of general formula (1) occurs. For the compounds of the present invention suitable solvents are solvents in which the compound of general formula (1) has a solubility of at least 10 g·l⁻¹, preferably of at least 20 g·l⁻¹, more preferably of at least 40 g·l⁻¹. Examples of suitable solvent classes in this respect are alcohols, alkanes, aryls, ethers, halogen-containing solvents, nitriles and the like, or mixtures thereof. Particularly suitable species are acetonitrile, diethyl ether, dioxane, ethanol, heptane, iso-propanol, methanol, methyl tert-butyl ether, octane, n-propanol, tetrahydrofuran and toluene but the skilled person will understand that solvents with structural similarity and comparable solubility will be equally suitable. Preferably the amount of compound of general formula (1) in the solid state is at least 5% by weight of the total weight of the mixture. More preferably this is from 5 to 95%, most preferably from 10 to 50%.

Racemization of said compound with general formula (1) can be effected by organic or inorganic bases with a pKₐ>9, preferably a pKₐ>12. Suitable examples are amines, such as NH₃, primary amines, secondary amines or tertiary amines; amidines, such as DBU or DBN; guanidines, such as TMG; metal hydroxides, such as LiOH, NaOH, KOH or CsOH; metal carbonates, such as Na₂CO₃, K₂CO₃, Cs₂CO₃; metal alcoholates, such as NaOMe, NaOEt, KOtBu; metal hydrides, such as NaH or CaH₂; metal amides, such as NaNH₂, LDA or KHMDS; or metal alkyls, such as BuLi, Et₂Zn or MeMgCl.

The process is preferably performed at temperatures between 0 and 160° C., more preferably between 20 and 120° C. The temperature may be kept constant, but the process can also be performed under temperature variations such as cyclic temperature variations.

The mechanical processing is effected by application of high sheer force or impact forces, for example by grinding. Preferably grinding is effected by stirring or milling or shaking or ultrasound in the presence of particles that are insoluble in the reaction mixture, for example wet milling, and/or by ultrasound and/or by using a mechanical stirring device such as a turbine stirrer, for instance a Rushton turbine stirrer, and/or by using a rotor mill, mortar mill, disc mill or ball mill, and/or by using an ultraturax mixer and/or by using an external loop containing a mill of high sheer pump. When stirring in the presence of insoluble particles is applied, said particles preferably have a diameter of from 0.2 mm to 5 cm and are made from glass and/or sand and/or ceramic and/or metal and/or other inert materials.

In one embodiment it was found that the deracemization time increases linearly with the amount of solids in the slurry. Furthermore, the time needed for the system to overcome the threshold of the autocatalytic process could be minimized by starting from an enantio-enriched solid phase. It is therefore beneficial to start with a small amount of solids having a high e.e., and then gradually feed the slurry with racemic material. In this way, the solid phase can sustain a high e.e., resulting in a high deracemization rate. Overall this shortens the time to reach an enantiopure solid phase.

Although the gradual feeding can be realized mechanically, in another embodiment the target molecule may advantageously be synthesized in situ during the process, making the practical execution very simple. To show the practical applicability without limiting the scope, the non-steroidal anti-inflammatory drug (S)-naproxen ((S)-2-(6-methoxynaphthalen-2-yl)propionic acid) is used as an example. Naproxen, as well as its sodium salt, crystallizes as a racemic compound thereby hampering a classical resolution. The methyl and ethyl ester of naproxen (1, R₁=2-(6-methoxynaphthalen-2-yl) and R₂═OCH₃ or OCH₂CH₃, respectively) however, crystallize as a racemic mixture or conglomerate, i.e. as separate enantiomorphous phases, and can easily be racemized in solution. However, poor e.e.-values have been obtained by Arai et al. (U.S. Pat. No. 4,417,070) through seeding of a clear saturated solution of methyl (RS)-2-(6-methoxynaphthalen-2-yl)propanoate with methyl (S)-2-(6-methoxynaphthalen-2-yl)propanoate and cooling the mixture to allow further crystal growth. This clearly indicates that the precondition of conglomerate and racemization as such is not sufficient. In U.S. Pat. No. 4,417,070 these preconditions are fulfilled, but the results obtained by the disclosed enrichment by crystallization from solution are nevertheless very poor and thus do not give an indication that the grinding process of the present invention will be a suitable alternative. Hence, even if the precondition of conglomerate behavior is met, this does not automatically result in a (practical) preferential crystallization as argued by G. Coquerel (Topics in Current Chemistry 2007, 269, 1-51).

Similar results are also obtained with the mono- and dibenzylamine salts of flurbiprofen (1, R₁=2-(2-fluorobiphenyl-4-yl) and R₂═NH₂CH₂Ph⁺ or NH(CH₂Ph₂)₂⁺, respectively). The asymmetric transformation of the diastereomeric salts of flurbiprofen with (−)-α-methylbenzylamine has been described in DE 2,809,794. Amides of flurbiprofen also are suitable substrates in the present invention since these are solids that can be easily isolated.

Interestingly, esters of general formula (1) wherein R₂ is OR₃ can be transformed into esters of general formula (1) wherein said group OR₃ is exchanged for a group OR₇ which is from the same genus as defined for OR₃ with the proviso that OR₃ and OR₇ are not the same, or wherein the said group OR₃ is exchanged for a group NR₄R₅.

For example, ethyl 2-(6-methoxynaphthalen-2-yl)propanoate can be transformed into methyl 2-(6-methoxynaphthalen-2-yl)propanoate under basic conditions using methanol as a solvent while the solubility of methyl 2-(6-methoxynaphthalen-2-yl)propanoate is lower in this solvent. These two properties can be utilized to generate a supersaturated solution of methyl 2-(6-methoxynaphthalen-2-yl)propanoate, starting from a saturated solution of ethyl 2-(6-methoxynaphthalen-2-yl)propanoate, thereby gradually feeding the slurry with racemic methyl 2-(6-methoxynaphthalen-2-yl)propanoate, without the necessity to cool the system. For example, a mixture of ethyl (RS)-2-(6-methoxynaphthalen-2-yl)propanoate and methyl (S)-2-(6-methoxynaphthalen-2-yl)propanoate in a ratio of 92:8 was partially dissolved in a solution of sodium methoxide in methanol, suitable concentrations of which are 1-25 wt %, preferably 5-15 wt %. The mixture is then subjected to an attrition-enhanced process, for instance by stirring with a magnetic stirring bar in the presence of glass beads. The result is a complete conversion of racemic ethyl 2-(6-methoxynaphthalen-2-yl)propanoate in an enantiomerically pure solid phase of methyl (S)-2-(6-methoxynaphthalen-2-yl)propanoate, with a deracemization time of methyl 2-(6-methoxynaphthalen-2-yl)propanoate that is reduced dramatically.

In yet another embodiment, preferred compounds of general formula (1) are:
(a) an ester of an α-aryl propionic acid and an alcohol R₃OH, or
(b) a salt of an α-aryl propionic acid and an alkaline or an alkaline earth metal or an amine of general formula NR₄R₅R₆, or
(c) an amide of an α-aryl propionic acid and an amine of formula HNR₄R₅;

Preferably the α-aryl propionic acid is one of the following: 2-(p-methylallylaminophenyl)propionic acid, 2-(4-chlorophenyl)-α-methyl-5-benzoxazoleacetic acid, 2-(8-methyl-10,11-dihydro-11-oxodibenz[b,f]oxepin-2-yl)propionic acid, 6-chloro-α-methyl-9H-carbazole-2-acetic acid, 2-(3-phenoxyphenyl)propionic acid, 2-(4-fluorophenyl)-α-methyl-5-benzoxazoleacetic acid, 2-(2-fluorobiphenyl-4-yl)propionic acid, 2-(4-isobutylphenyl)propionic acid, 2-[4-(1-oxo-2-isoindolinyl)phenyl]propionic acid, 2-(3-benzoylphenyl)propionic acid, α-methyl-4-[(2-oxocyclopentyl)methyl]benzeneacetic acid, 2-(6-methoxynaphthalen-2-yl)propionic acid, 3-chloro-4-(2,5-dihydro-1H-pyrrol-1-yl)-α-methylbenzeneacetic acid, 2-(5H-[1]benzopyrano[2,3-b]pyridin-7-yl)propionic acid, α-methyl-4-(2-thienylcarbonyl)benzeneacetic acid, 2-(4-cyclohexyl-1-naphthyl)propionic acid, 2-[4-(3-oximinocyclohexyl)phenyl]propionic acid and 2-(10,11-dihydro-10-oxodibenzo[b,f]thiepin-2-yl)propionic acid.

The alcohol R₃OH may be any alcohol suitable for the protection of carboxylic acids. Suitable examples are allyl alcohol, 9-anthrylmethyl alcohol, benzyl alcohol, benzyloxymethyl alcohol, p-bromobenzyl alcohol, p-bromophenacyl alcohol, 3-buten-1-yl alcohol, n-butanol, sec-butanol, t-butanol, 2-(t-butyldimethylsilyl)ethyl alcohol, 2-(di-t-butylmethylsilyl)ethyl alcohol, 2-(t-butyldiphenylsilyl)ethyl alcohol, cyclohexanol, carboxamidomethyl alcohol, cinnamyl alcohol, cyclopentanol, cyclopropylmethyl alcohol, 5-dibenzosuberyl alcohol, 2,6-dichlorobenzyl alcohol, 2,2-dichloro-1,1-difluoroethanol, 2,6-dimethoxybenzyl alcohol, 4-(dimethylaminocarbonyl)benzyl alcohol, 2,6-dimethylbenzyl alcohol, 1,1-dimethylpropanol, 1,2-dimethylpropanol, 2,2-dimethylpropanol, 2-(dimethylthiophosphinyl)ethyl alcohol, 2-(9,10-dioxo)anthrylmethyl alcohol, diphenylmethyl alcohol, 2-(diphenylphosphino)ethyl alcohol, 1,3-dithianyl-2-methyl alcohol, ethanol, 9-fluorenylmethyl alcohol, 2-haloethanol, isobutanol, isopropanol, 2-(isopropyldimethylsilyl)ethyl alcohol, p-methoxybenzyl alcohol, methoxyethoxyethyl alcohol, methoxyethyl alcohol, p-methoxyphenacyl alcohol, methanol, 1-methylbutanol, 2-methylbutanol, 3-methylbutanol, methylcarbonylethyl alcohol, α-methylcinnamyl alcohol, p-(methylmercapto)phenyl alcohol, α-methylphenacyl alcohol, 1-methyl-1-phenylethyl alcohol, 4-(methylsulfinyl)benzyl alcohol, methylthiomethyl alcohol, 2-methylthioethyl alcohol, o-nitrobenzyl alcohol, p-nitrobenzyl alcohol, bis(o-nitrophenyl)methyl alcohol, 2-(p-nitrophenylsulfenyl)ethyl) alcohol, n-pentanol, phenacyl alcohol, phenyl alcohol, phenyldimethylsilyl alcohol, N-phthalimidomethyl alcohol, 4-picolyl alcohol, piperonyl alcohol, propanol, 1-pyrenylmethyl alcohol, 2-(2′-pyridyl)ethyl alcohol, 4-sulfobenzyl alcohol, 2-tetrahydrofuranyl alcohol, 2-tetrahydropyranyl alcohol, 2-(p-toluenesulfonyl)ethyl alcohol, 2,2,2-trichloroethanol, triethylsilyl alcohol, 2-(trifluoromethyl)-6-chromylmethyl alcohol, 2,4,6-trimethylbenzyl alcohol, 4-(trimethylsilyl)-2-buten-1-yl alcohol, trimethylsilyl alcohol, 2-(trimethylsilyl)ethyl alcohol, 2-(trimethylsilyl)ethoxymethyl alcohol and triphenylmethyl alcohol.

Suitable examples of amines are those wherein R₄, R₅ and R₆ are independently benzyl, butyl, ethyl, hydrogen, 2-hydroxyethyl, iso-propyl, methyl, p-nitrophenyl, phenyl, 1-phenylethyl, 2-phenylethyl, propyl or wherein R₄ and R₅ are in a ring structure to form morpholino, piperidino or pyrrolidino. Most preferably the compound of general formula (1) is the methyl ester or the ethyl ester of 2-(6-methoxynaphthalen-2-yl)propionic acid or an amide or salt of 2-(2-fluorobiphenyl-4-yl)propionic acid.

In another embodiment the optically pure esters are converted to carboxylic acids of general formula (2)

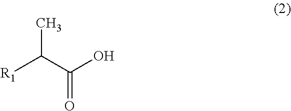

having an enantiomeric excess of from 50 to 99.99%, preferably from 90 to 99,99%, and wherein R₁ is as defined above by means of hydrolysis.

In a second aspect of the invention the compounds of general formula (2) are used for the preparation of a medicament. Suitable examples are the anti-inflammatory drugs (S)-2-(2-fluorobiphenyl-4-yl)propionic acid, (R)-2-(2-fluorobiphenyl-4-yl)propionic acid, (S)-2-(4-isobutylphenyl)propionic acid, (S)-2-(6-methoxynaphthalen-2-yl)propionic acid and (S)-2-(3-benzoylphenyl)propionic acid, or a salt of these compounds.

### LEGEND TO THE FIGURES

FIG. 1 shows the evolution of the solid phase enantiomeric ratio between the (R)- and the (S)-enantiomer of methyl 2-(6-methoxynaphthalen-2-yl)propanoate under grinding conditions, showing an exponential increase in the solid phase enantiomeric excess. The Y-axis represents the enantiomeric ratio (%), the X-axis represents the time (days); solid bars (black) represent methyl (R)-2-(6-methoxynaphthalen-2-yl)propanoate, open bars (white) represent methyl (S)-2-(6-methoxynaphthalen-2-yl)propanoate.

FIG. 2 shows the evolution of the solid phase enantiomeric excess in the (S)-enantiomer of methyl 2-(6-methoxynaphthalen-2-yl)propanoate (filled squares) and ethyl 2-(6-methoxynaphthalen-2-yl)propanoate (filled diamonds) during the esterification mediated deracemization under grinding conditions. The fraction of methyl 2-(6-methoxynaphthalen-2-yl)propanoate in the solid phase is depicted by the open circles. The Y-axis represents the solid phase enantiomeric excess (%), or the molar fraction (%), respectively, and the X-axis represents the time (hours).

### EXAMPLES

### Example 1

### Methyl (S)-2-(6-methoxynaphthalen-2-yl)propanoate

To a solution of (S)-2-(6-methoxynaphthalen-2-yl)propanoic acid (naproxen, 6.13 g, 27.0 mmol) in methanol (250 mL) was added 35 drops of concentrated H₂SO₄ and the reaction mixture was stirred for overnight before it was diluted with CH₂Cl₂ (approx. 50 mL), washed with an aqueous saturated NaHCO₃ solution and dried over Na₂SO₄. Solvent evaporation gave the title product. ¹H NMR (400 MHz, CDCl₃): δ=7.62 (s, 1H), 7.57 (d, 1H, J=8.5 Hz), 7.48-7.45 (m, 2H), 7.18 (d, 1H, J=2.6 Hz), 6.89 (d, 1H, J=2.4 Hz), 3.71 (q, 1H, J=7.1 Hz), 3.36 (s, 3H), 3.28 (s, 3H), 1.52 (d, 3H, J=7.1 Hz).

### Example 2

### Methyl (RS)-2-(6-methoxynaphthalen-2-yl)propanoate

To a solution of (RS)-2-(6-methoxynaphthalen-2-yl)propanoic acid (14.25 g, 62.8 mmol) in methanol (450 mL) was added 70 drops of concentrated H₂SO₄ and the reaction mixture was stirred for overnight before it was diluted with CH₂Cl₂ (approx. 1 L), washed with aqueous saturated NaHCO₃ and dried over Na₂SO₄. Solvent evaporation gave the title methyl ester quantitatively. ¹H NMR (400 MHz, CDCl₃): δ=7.62 (s, 1H), 7.57 (d, 1H, J=8.5 Hz), 7.48-7.45 (m, 2H), 7.18 (d, 1H, J=2.6 Hz), 6.89 (d, 1H, J=2.4 Hz), 3.71 (q, 1H, J=7.1 Hz), 3.36 (s, 3H), 3.28 (s, 3H), 1.52 (d, 3H, J=7.1 Hz).

### Example 3

### Ethyl (RS)-2-(6-methoxynaphthalen-2-yl)propanoate

Following the procedure of Example 1, however with ethanol instead of methanol, (RS)-2-(6-methoxynaphthalen-2-yl)propanoic acid (naproxen, 5.74 g, 27.0 mmol) in 250 mL ethanol with approx. 40 drops concentrated H₂SO₄ was converted to the title product quantitatively. ¹H NMR (300 MHz, CDCl₃): δ=7.72-7.67 (m, 3H), 7.41 (dd, 1H, J=1.8 Hz, J=8.4 Hz), 7.16-7.12 (m, 2H), 4.21-4.05 (m, 2H), 3.91 (s, 3H), 3.83 (q, 3H, J=7.2 Hz), 1.55 (d, 2H, J=3.0 Hz), 1.20 (t, 3H, J=8.1 Hz).

### Example 4

### Deracemization of methyl (RS)-2-(6-methoxynaphthalen-2-yl)propanoate

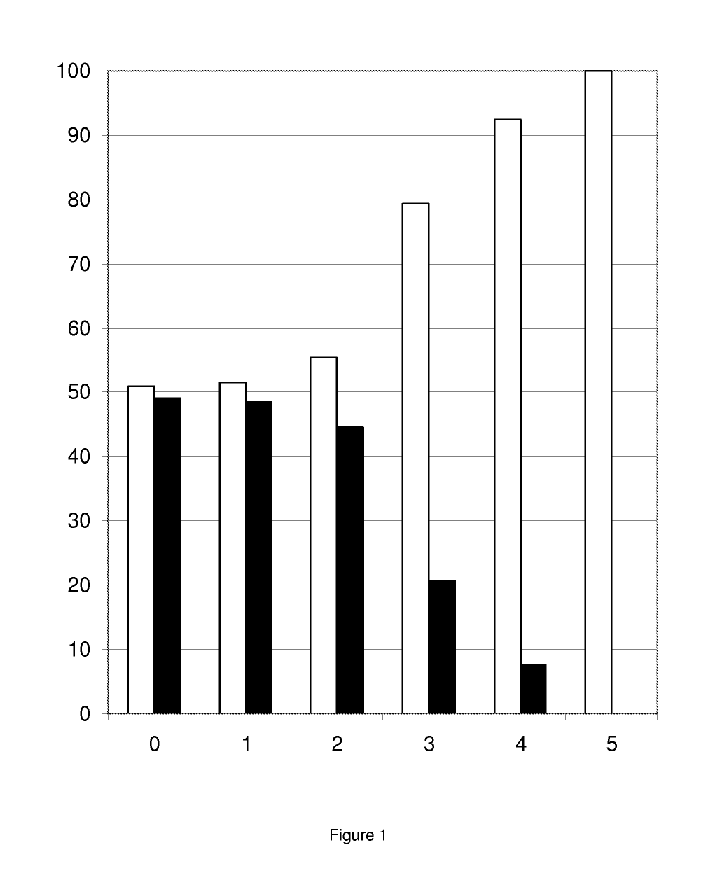

In a standard 10 mL sample vial were added glass beads (Ø 2-2.5 mm, Aldrich, 8.7 g), methyl (RS)-2-(6-methoxynaphthalen-2-yl)propanoate (0.7553 g), methyl (S)-2-(6-methoxynaphthalen-2-yl)propanoate (0.0030 g) and NaOMe/MeOH (6.302 g from a stock solution prepared by dissolving 2.2 g Na in 45 mL MeOH). The sample vial was closed with a septum, and placed on an Elma Transsonic T470/H ultrasonic bath. The bath was kept at a constant temperature of 23° C. using a cooling spiral that was attached to a Julabo F25 thermostat bath. For sampling, 0.3 mL of the slurry was taken using a syringe, filtered on a P4 glass filter and washed with MeOH (approx. 2 mL). The chiral purity was measured using chiral HPLC. ¹H NMR (400 MHz, CDCl₃): δ=7.62 (s, 1H), 7.57 (d, 1H, J=8.5 Hz), 7.48-7.45 (m, 2H), 7.18 (d, 1H, J=2.6 Hz), 6.89 (d, 1H, J=2.4 Hz), 3.71 (q, 1H, J=7.1 Hz), 3.36 (s, 3H), 3.28 (s, 3H), 1.52 (d, 3H, J=7.1 Hz). HPLC analysis was performed on Chiralcel-OJ (250×4.6 mm ID) column, eluent n-hexane/2-propanol 98/2 v/v %, flow 1 mL·min⁻¹, room temperature, detection at λ=254 nm. Retention times methyl (S)-2-(6-methoxynaphthalen-2-yl)propanoate 10.5 min, methyl (R)-2-(6-methoxynaphthalen-2-yl)propanoate 11.4 min. The results of this experiment are given in FIG. 1. It can be seen from this Figure that already an initial enantiomeric excess of 1.5% results in the exponential evolution to an enantiopure methyl (S)-2-(6-methoxynaphthalen-2-yl)propanoate solid phase.

### Example 5

### Esterification mediated deracemization of methyl (RS)-2-(6-methoxynaphthalen-2-yl)propanoate

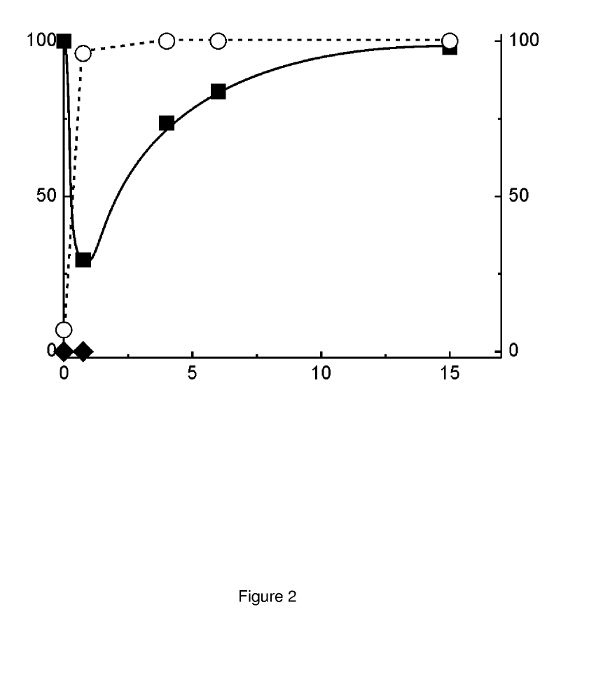

Under Schlenck conditions, to a 25 mL round bottom flask were added glass beads (8.7 g), ethyl (RS)-2-(6-methoxynaphthalen-2-yl)propanoate (0.6120 g), methyl (S)-2-(6-methoxynaphthalen-2-yl)propanoate (0.050 g) and MeOH/MeOH (6.502 g from a stock containing 10 mL MeOH and 0.5 g Na) and an oval magnetic stirring bar. The process was started by stirring at 700 rpm. For sampling, 0.3 mL of the slurry was taken, filtered on a P4 glass filter and washed with approx. 2 mL MeOH. The chiral purity was measured using chiral HPLC. ¹H NMR (400 MHz, CDCl₃): δ=7.62 (s, 1H), 7.57 (d, 1H, J=8.5 Hz), 7.48-7.45 (m, 2H), 7.18 (d, 1H, J=2.6 Hz), 6.89 (d, 1H, J=2.4 Hz), 3.71 (q, 1H, J=7.1 Hz), 3.36 (s, 3H), 3.28 (s, 3H), 1.52 (d, 3H, J=7.1 Hz). HPLC analysis was performed on Chiralcel-OJ (250×4.6 mm ID) column, eluent n-hexane/2-propanol 98/2 v/v %, flow 1 mL·min⁻¹, r.t., detection λ=254 nm. Retention times methyl (S)-2-(6-methoxynaphthalen-2-yl)propanoate 10.5 min, methyl (R)-2-(6-methoxynaphthalen-2-yl)propanoate 11.4 min. The results of this experiment are given in FIG. 2.

### Example 6

### (RS)-2-(2-fluorobiphenyl-4-yl)propionic acid benzylamine salt

To a solution of (RS)-2-(2-fluorobiphenyl-4-yl)propionic acid (flurbiprofen, 4.89 g, 20 mmol) in 19 mL of ethanol was slowly added 2.20 g (20.5 mmol) of benzylamine. After standing for 15 min the clear solution was seeded with 2-(2-fluorobiphenyl-4-yl)propionic acid benzylamine salt. After 4 hours the crystals were filtered, washed with 10 mL of toluene and dried, yielding 5.95 g (85%) of the title compound as a white crystalline solid. ¹H NMR (300 MHz, CDCl₃): δ=7.47-7.19 (m, 14H), 7.03-7.01 (2×s, 2H), 3.70 (s, 2H), 3.41 (q, 1H), 1.31 (d, 3H). According to second harmonic generation measurement and X-ray powder diffraction this salt is a racemic conglomerate.

### Example 7

### (R)-2-(2-fluorobiphenyl-4-yl)propionic acid benzylamine salt

To a solution of (R)-2-(2-fluorobiphenyl-4-yl)propionic acid (490 mg, 2.0 mmol) in 3 mL of ethanol was slowly added 218 mg (2.05 mmol) of benzylamine in 1 mL of ethanol. After standing for 30 minutes the clear solution slowly started crystallizing. After 3 days the crystals were filtered, washed with 1 mL of ethanol, and dried. This resulted in 423 mg (60%) of the title compound as a white crystalline solid. From the filtrate additional 120 mg of product was isolated after partial evaporation of the solvent (total yield 77%).

### Example 8

### (RS)-2-(2-fluorobiphenyl-4-yl)propionic acid dibenzylamine salt

To a solution of (RS)-2-(2-fluorobiphenyl-4-yl)propionic acid (244 mg, 1.0 mmol) in 1 mL of ethanol was added 192 μL (197 mg, 1.0 mmol) of dibenzylamine. After crystallization of the clear solution the crystals were filtered off, washed with a minimal amount of ethanol and dried. This yielded 400 mg (91%) of the title compound as a white crystalline solid. ¹H NMR (300 MHz, CDCl₃): δ=7.85 (br s, 2H), 7.53-7.12 (m, 18H), 3.78 (s, 4H), 3.45 (q, 1H), 1.48 (d, 3H). According to second harmonic generation measurement and X-ray powder diffraction this salt is a racemic conglomerate.

### Example 9

### (R)-2-(2-fluorobiphenyl-4-yl)propionic acid dibenzylamine salt

To a solution of (R)-2-(2-fluorobiphenyl-4-yl)propionic acid (490 mg, 2.0 mmol) in 3 mL of ethanol was added 400 mg (2.0 mmol) of dibenzylamine in 1 mL of ethanol. After the addition the clear solution crystallized. After 3 days the crystals were filtered, washed with 1 mL of ethanol and dried. This yielded 754 mg (86%) of the title compound as a white crystalline solid.

### Example 10

### Racemization of (R)-2-(2-fluorobiphenyl-4-yl)propionic acid benzylamine salt using 1,1,3,3-tetramethylguanidine

To a standard 25 mL round bottom flask were added 0.0145 g of (R)-2-(2-fluorobiphenyl-4-yl)propionic acid benzylamine salt, 2.0 g toluene and 0.34 g 1,1,3,3-tetramethylguanidine. The mixture was heated to 100° C. Samples of the liquid were taken in time and analyzed using chiral HPLC. HPLC analysis was performed on AD-H Chiralpak (250×4.6 mm ID) column, eluent n-hexane/2-propanol 95/5 v/v %, flow 1 mL·min⁻¹, room temperature, detection at λ=254 nm. Retention times (R)-2-(2-fluorobiphenyl-4-yl)propionic acid benzylamine salt 14.8 min, (S)-2-(2-fluorobiphenyl-4-yl)propionic acid benzylamine salt 19.5 min. After 4 hours the enantiomeric excess was reduced to 6% ee.

### Example 11

### Racemization of (R)-2-(2-fluorobiphenyl-4-yl)propionic acid benzylamine salt

To a standard 25 mL round bottom flask were added 0.0130 g of (R)-2-(2-fluorobiphenyl-4-yl)propionic acid benzylamine salt, 2.0 g toluene and 0.18 g benzylamine. The mixture was heated to 80° C. Samples of the liquid were taken in time and enantiomeric excess was determined using chiral HPLC analysis. After 48 hours the enantiomeric excess was reduced to 69% ee, demonstrating the solution phase racemization.

### Example 12

### Deracemization of (RS)-2-(2-fluorobiphenyl-4-yl)propionic acid benzylamine salt

To a standard 50 mL round bottom flask were added 200 mg of (RS)-2-(2-fluorobiphenyl-4-yl)propionic acid benzylamine salt, 10 g of glass beads (Ø 2.5 mm), 20 mL of n-octane and an oval magnetic stirring bar. Stirring at 1000 rpm was started and the mixture was heated to 100° C. To the white suspension 50 mg (0.47 mmol, 7.5 mol % on total amount of salt) of benzylamine was added. To the resulting opaque (saturated) solution additional 1.80 g of (RS)-2-(2-fluorobiphenyl-4-yl)propionic acid benzylamine salt and 190 mg of (R)-2-(2-fluorobiphenyl-4-yl)propionic acid benzylamine salt were added (in total 2.19 g (6.2 mmol) of salt with starting e.e. of 9%). The thick suspension was stirred at 1000 rpm and 100° C. for additional 4 days. After cooling to ambient temperature the glass beads were sieved and the remaining off-white suspension was filtered on a P3 glass filter. This resulted in 1.92 g (88%) of (R)-2-(2-fluorobiphenyl-4-yl)propionic acid benzylamine salt (purity 93%).

1. Method for the synthesis of an α-aryl propionic acid derivative of general formula (1) 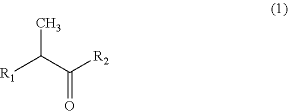
having an enantiomeric excess of from 50 to 99.99%, wherein R₁ is substituted or unsubstituted biphenyl, naphthyl, phenyl or thienyl and wherein R₂ is an amide or OR₃ with R₃ being a carboxyl protecting group, a substituted or unsubstituted amine cation or a metal cation, comprising subjecting a compound of general formula (1) having an enantiomeric excess of from 0 to 50% and wherein R₁ and R₂ are as defined above to mechanical processing, characterized in that said compound of general formula (1) having an enantiomeric excess of from 0 to 50% is present both in the solid state and in solution in a solvent and that said mechanical processing is effected by high sheer forced or impact forces.

2. Method according to claim 1 wherein said mechanical processing is grinding.

3. Method according to claim 1 wherein the amount of said compound of general formula (1) having an enantiomeric excess of from 0 to 50% present in the solid state is at least 5% by weight of the total weight of the mixture.

4. Method according to claim 1 wherein said compound of general formula (1) is
(a) an ester of an α-aryl propionic acid and an alcohol R₃OH, or
(b) a salt of an α-aryl propionic acid and an alkaline or an alkaline earth metal or an amine of general formula NR₄R₅R₆, or
(c) an amide of an α-aryl propionic acid and an amine of formula HNR₄R₅;
wherein said α-aryl propionic acid is chosen from the list consisting of 2-(p-methylallylaminophenyl)propionic acid, 2-(4-chlorophenyl)-□-methyl-5-benzoxazoleacetic acid, 2-(8-methyl-10,11-dihydro-11-oxodibenz[b,f]oxepin-2-yl)propionic acid, 6-chloro-α-methyl-9H-carbazole-2-acetic acid, 2-(3-phenoxyphenyl)propionic acid, 2-(4-fluorophenyl)-α-methyl-5-benzoxazoleacetic acid, 2-(2-fluorobiphenyl-4-yl)propionic acid, 2-(4-isobutylphenyl)propionic acid, 2-[4-(1-oxo-2-isoindolinyl)phenyl]propionic acid, 2-(3-benzoylphenyl)propionic acid, α-methyl-4-[(2-oxocyclopentyl)methyl]benzeneacetic acid, 2-(6-methoxynaphthalen-2-yl)propionic acid, 3-chloro-4-(2,5-dihydro-1H-pyrrol-1-yl)-α-methylbenzeneacetic acid, 2-(5H-[1]benzopyrano[2,3-b]pyridin-7-yl)propionic acid, α-methyl-4-(2-thienylcarbonyl)benzeneacetic acid, 2-(4-cyclohexyl-1-naphthyl)propionic acid, 2-[4-(3-oximinocyclohexyl)phenyl]-propionic acid and 2-(10,11-dihydro-10-oxodibenzo[b,f]thiepin-2-yl)propionic acid, and wherein said alcohol R₃OH is chosen from the list consisting of allyl alcohol, 9-anthrylmethyl alcohol, benzyl alcohol, benzyloxymethyl alcohol, p-bromobenzyl alcohol, p-bromophenacyl alcohol, 3-buten-1-yl alcohol, n-butanol, sec-butanol, t-butanol, t-butyldimethylsilyl alcohol, di-t-butylmethylsilyl alcohol, t-butyldiphenylsilyl alcohol, cyclohexanol, carboxamidomethyl alcohol, cinnamyl alcohol, cyclopentanol, cyclopropylmethyl alcohol, 5-dibenzosuberyl alcohol, 2,6-dichlorobenzyl alcohol, 2,2-dichloro-1,1-difluoroethanol, 2,6-dimethoxybenzyl alcohol, 4-(dimethylaminocarbonyl)benzyl alcohol, 2,6-dimethylbenzyl alcohol, 1,1-dimethylpropanol, 1,2-dimethylpropanol, 2,2-dimethylpropanol, dimethylthiophosphinyl alcohol, 2-(9,10-dioxo)anthrylmethyl alcohol, diphenylmethyl alcohol, 2-(diphenylphosphino)ethyl alcohol, 1,3-dithianyl-2-methyl alcohol, ethanol, 9-fluorenylmethyl alcohol, 2-haloethanol, isobutanol, isopropanol, isopropyldimethylsilyl alcohol, p-methoxybenzyl alcohol, methoxyethoxymethyl alcohol, methoxymethyl alcohol, p-methoxyphenacyl alcohol, methanol, 1-methylbutanol, 2-methylbutanol, 3-methylbutanol, methyl carbonyl alcohol, α-methylcinnamyl alcohol, p-(methylmercapto)phenyl alcohol, α-methylphenacyl alcohol, 1-methyl-1-phenylethyl alcohol, 4-(methylsulfinyl)benzyl alcohol, methylthiomethyl alcohol, 2-methylthioethyl alcohol, o-nitrobenzyl alcohol, p-nitrobenzyl alcohol, bis(o-nitrophenyl)methyl alcohol, 2-(p-nitrophenylsulfenyl)ethyl) alcohol, n-pentanol, phenacyl alcohol, phenyl alcohol, phenyldimethylsilyl alcohol, N-phthalimidomethyl alcohol, 4-picolyl alcohol, piperonyl alcohol, propanol, 1-pyrenylmethyl alcohol, 2-(2′-pyridyl)ethyl alcohol, 4-sulfobenzyl alcohol, 2-tetrahydrofuranyl alcohol, 2-tetrahydropyranyl alcohol, 2-(p-toluenesulfonyl)ethyl alcohol, 2,2,2-trichloroethanol, triethylsilyl alcohol, 2-(trifluoromethyl)-6-chromylmethyl alcohol, 2,4,6-trimethylbenzyl alcohol, 4-(trimethylsilyl)-2-buten-1-yl alcohol, trimethylsilyl alcohol, 2-(trimethylsilyl)ethyl alcohol, 2-(trimethylsilyl)ethoxymethyl alcohol and triphenylmethyl alcohol, and
wherein R₄ and R₅ are independently benzyl, ethyl, hydrogen, 2-hydroxyethyl, iso-propyl, methyl, p-nitrophenyl, phenyl, 1-phenylethyl, 2-phenylethyl, propyl or wherein R₄ and R₅ are in a ring structure to form morpholino, piperidino or pyrrolidino.

5. Method according to claim 4 wherein said compound of general formula (1) is the methyl ester or the ethyl ester of 2-(6-methoxynaphthalen-2-yl)propionic acid or an amide or salt of 2-(2-fluorobiphenyl-4-yl)propionic acid.

6. Method according to claim 2 wherein said grinding is effected by stirring, milling, shaking or ultrasound in the presence of particles that are insoluble in the reaction mixture and/or by using a turbine and/or by using an ultraturax mixer.

7. Method according to claim 6 wherein said particles have a diameter of from 0.2 mm to 5 cm.

8. Method according to claim 6 wherein said particles are glass, sand, ceramic and/or metal particles.

9. Method according to claim 1 wherein said group OR₃ is exchanged for a group OR₇ which is from the same genus as defined for OR₃ with the proviso that OR₃ and OR₇ are not the same, or wherein the said group OR₃ is exchanged for a group NR₄R₅.

10. Method according to claim 9 wherein OR₃ is ethyl and OR₇ is methyl or wherein OR₃ is methyl and OR₇ is ethyl, and R₁ is 2-(6-methoxynaphthalen-2-yl).

11. Method according to claim 1 further comprising hydrolysis to give a compound of general formula (2), or a salt thereof, 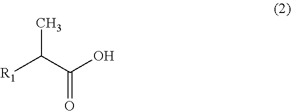
having an enantiomeric excess of from 50 to 99.99% and wherein R₁ is as defined above.

12. Use of (S)-2-(2-fluorobiphenyl-4-yl)propionic acid or (R)-2-(2-fluorobiphenyl-4-yl)propionic acid or (S)-2-(4-isobutylphenyl)propionic acid or (S)-2-(6-methoxynaphthalen-2-yl)propionic acid or (S)-2-(3-benzoylphenyl)propionic acid prepared according to the method of claim 11 in the preparation of a medicament.
